(19) United States
(12) Patent Application Publication
(10) Pub. No.: US 2014/0316493 A1
(43) Pub. Date: Oct. 23, 2014

(54) Lumbar Cryotherapy Belt with Access Window

(71) Applicant: Bryan E. Kilbey, DeFuniak Sprs, FL (US)

(72) Inventor: Bryan E. Kilbey, DeFuniak Sprs, FL (US)

(21) Appl. No.: 13/865,271

(22) Filed: Apr. 18, 2013

(51) Int. Cl. A61F 7/10

(52) U.S. Cl. CPC A61F 7/10; A61F 2007/0026
USPC 607/108

(57) ABSTRACT

The present invention comprises a cryo therapy system suitable for use on a patient's lumbar area. A belt is provided with a front closure and a posterior window. The belt is looped around the patient's abdomen, with the posterior window lying over the lumbar spine. The front closure is secured using VELCRO fasteners. A pocket is preferably provided on each end of the belt. The pockets allow the user to more easily apply and tighten the belt. A cover panel is provided to selectively cover and close the posterior window in the belt. The cover panel includes an inward facing surface which is at least partially covered in loop material. One or more cold packs are provided. Each cold pack includes a hook panel configured to engage the loop material on the inward facing surface of the cover panel.

### CROSS-REFERENCES TO RELATED APPLICATIONS

This application is a non-provisional application claiming priority to U.S. Provisional Application No. 61/525,812 tiled on Apr. 18, 2012.

### STATEMENT REGARDING FEDERALLY SPONSORED RESEARCH OR DEVELOPMENT

Not Applicable.

### MICROFICHE APPENDIX

Not Applicable.

### BACKGROUND OF THE INVENTION

1. Field of the Invention

This invention relates to the field of medical products. More specifically, the invention comprises a lumbar cryotherapy system which allows convenient access to a surgical site for placement of cryotherapy devices and other purposes.

2. Description of the Related Art

The minimization of swelling following a surgical procedure is a common goal. One method is to employ “cold therapy,” which is also known as “cryo therapy.” Cryo therapy assumes a variety of forms—ranging from simple ice or gel packs to sophisticated chilled water circulating devices. These devices must provide sufficient cooling to minimize swelling yet not cool the tissue to the point where damage might occur.

The simplest form of cryo therapy involves placing a sealed bag of ice against the affected portion of the patient and retaining it in position using a compressive wrap. Some type of insulating layer is typically placed next to the patient's skin in order to prevent the ice pack from overcooling the tissue it lies proximate to. The use of ice is somewhat risky in this situation. Although ice can absorb a significant amount of heat—owing to the phase change from a solid to a liquid—its melting temperature is lower than would be ideal. During the phase change an ice pack can cool adjacent tissue to near the freezing point of water. A temperature this low can cause tissue damage.

Gel packs reduce this concern by eliminating the phase change. Gel packs do not freeze when placed in a typical freezer. They emerge instead as very cold liquids. When they are applied to the patient, no phase change is produced. Thus, as those skilled in the art will know, the adjacent tissue is able to warm the gel above its initial temperature in a fairly short time. Of course, the lack of a phase change means that the gel pack cannot absorb nearly as much heat as a cooling medium which employs a phase change.

The preferred cooling medium would therefore employ a phase change. However, it would also have a freezing temperature significantly above the freezing point of water in order to minimize the risk of tissue damage. The preferred cooling medium is one that freezes between about 5 degrees Celsius and about 20 degrees Celsius. Such a medium can absorb considerable heat via the phase change from a solid to a liquid. In addition, such a cooling medium poses little risk of tissue damage (as its phase change temperature is above 0 degrees Celsius). Although the present invention can function with a wide variety of cooling media, it preferably uses a cooling medium with a freezing point between about 5 degrees and about 20 degrees Celsius.

One common application for cryo therapy is in post-surgical swelling reduction for lumbar spinal surgery. It is known to use a compressive “belt” to hold cold packs in position over the patient's lumbar spine. The cold packs used for this purpose must be changed every few hours. Thus, the device used to hold the cold packs in position must be regularly loosened and then reapplied. This has traditionally been a cumbersome process. The present invention alleviates this concern by providing convenient access to the lumbar surgical site. The invention also includes features allowing it to be easily installed and adjusted.

### BRIEF SUMMARY OF THE PRESENT INVENTION

The present invention comprises a cryo therapy system suitable for use on a patient's lumbar area. A belt is provided with a front closure and a posterior window. The belt is looped mound the patient's abdomen, with the posterior window lying over the lumbar spine. The front closure is secured using VELCRO fasteners. A pocket is preferably provided on each end of the belt. The pockets allow the user to more easily apply and tighten the belt.

A cover panel is provided to selectively cover and close the posterior window in the belt. The cover panel attaches to the belt using VELCRO features. The cover panel preferably also includes adjustable straps so that it may be selectively tightened in position. The cover panel includes an inward facing surface which is at least partially covered in loop material. One or more cold packs are provided. Each cold pack includes a hook panel configured to engage the loop material on the inward facing surface of the cover panel.

A cold pack may be placed on the patient without having to remove the belt. The patient is rolled onto his or her side. One side of the cover panel may then be detached from the belt, which allows the cover panel to swing away from the posterior window like a door. The patient's lumbar area is thereby exposed. One or more cold packs may then b attached to the inward facing surface of the cover panel by pressing the hook panel(s) on the cold pack against the mop material on the cover panel. The loose side of the cover panel is then reattached to the belt in order to close the posterior window. The cold pack is thereby held against the patient's lumbar area.

Cold packs may be placed and exchanged at desired intervals by opening the cover panel. This task may easily be performed without disturbing the belt itself. Other features are preferably provided to allow adjustment of the belt as the condition of a patient's abdomen changes. For example, an abdominal pad may be provided on the inward facing portion of the anterior section of the belt. The abdominal pad is preferably attached to the belt using VELCRO material so that it may be easily positioned. Abdominal pads of different sized and/or shapes are preferably provided to accommodate changes in patient geometry.

### BRIEF DESCRIPTION OF THE SEVERAL VIEWS OF THE DRAWINGS

FIG. 1 is a perspective view, showing the belt portion of the present invention.

FIG. 2 is a perspective view, showing the opposite side of the belt of FIG. 1.

FIG. 3 is a perspective view, showing a cover panel which is used in conjunction with the belt.

FIG. 4 is an exploded perspective view, showing the inclusion of a lumbar stay in the cover panel.

FIG. 5 is a perspective view, showing the lumbar panel from the opposite side.

FIG. 6 is a perspective view, showing the cover panel and belt assembled together.

FIG. 7 is a detailed perspective view, showing how the inclusion of finger and thumb pockets on the belt aids the patient in installing the belt.

FIG. 8 is a perspective view, showing a patient applying the belt.

FIG. 9 is a perspective view, showing a patient applying the belt.

FIG. 10 is a perspective view, showing the cover panel being applied to the belt.

FIG. 11 is a perspective view, showing the belt secured in position on a patient.

FIG. 12 is a perspective view, showing the belt being applied with an abdominal pad.

FIG. 13 is a perspective view, showing a patient placed in a position where the cover panel may be opened with the belt still in place.

FIG. 14 is a perspective view, showing the cover panel opened to reveal the window in the belt.

FIG. 15 is a perspective view, showing a pair of cold packs.

FIG. 16 is a perspective view, showing a cold pack of FIG. 15 being applied to the cover panel.

REFERENCE NUMERALS IN THE DRAWINGS

| | |
|---|---|
| 10 | belt |
| 12 | top edge |
| 14 | bottom edge |
| 16 | right thumb pocket |
| 18 | right finger pocket |
| 20 | left thumb pocket |
| 22 | left finger pocket |
| 24 | tension strap |
| 26 | tension strap |
| 28 | tension strap |
| 30 | tension strap |
| 32 | window |
| 34 | loop panel |
| 36 | 4-way stretch panel |
| 38 | cover panel |
| 40 | main body |
| 42 | stay pocket cover |
| 44 | strap loop |
| 46 | strap loop |
| 48 | strap loop |
| 50 | strap loop |
| 54 | lumbar stay |
| 56 | stay pocket |
| 58 | hook tab |
| 60 | inward facing surface |
| 62 | left hook panel |
| 64 | right hook panel |
| 66 | assembly |
| 68 | hand |
| 70 | left hand |
| 72 | right hand |
| 73 | abdominal pad |
| 74 | hook panel |
| 76 | hook panel |
| 78 | left edge |
| 80 | right edge |
| 82 | soft surface |
| 112 | cold pack |
| 114 | vent |
| 116 | relief |
| 118 | hook panel |

### DETAILED DESCRIPTION OF THE INVENTION

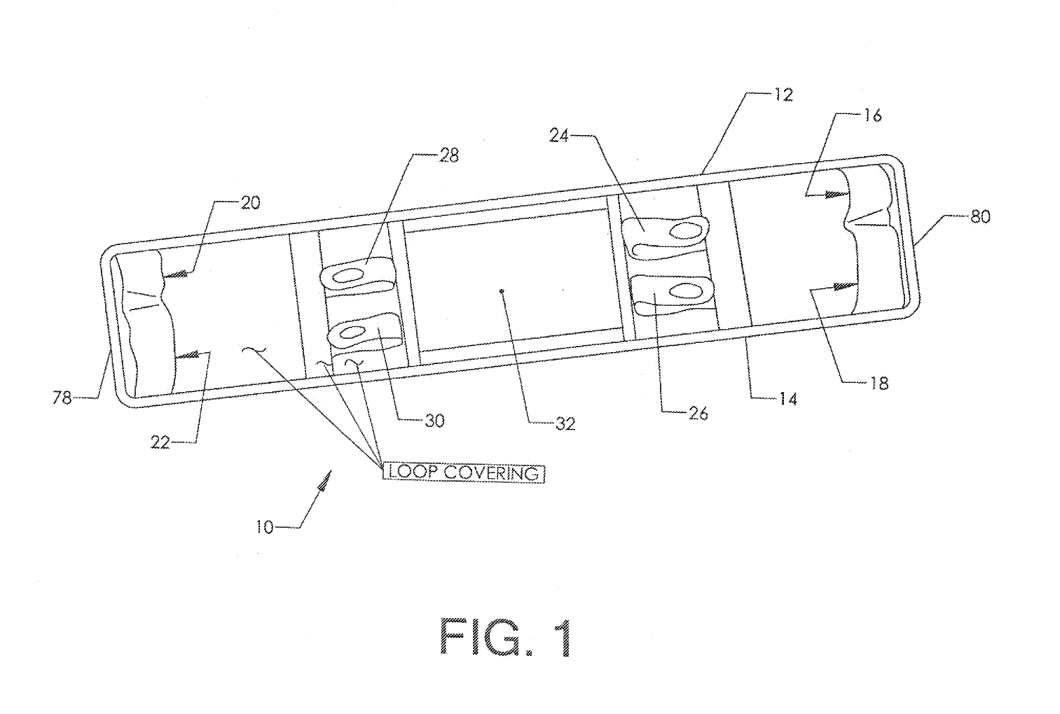

The present invention uses two main components a belt and a cover panel. FIG. 1 shows belt 10. Belt 10 has an inward facing side (which normally faces the patient) and an outward facing side. FIG. 1 shows the outward facing side. The belt is generally rectangular, being bounded by top edge 12, bottom edge 14, right edge 80 and left edge 78. A substantial portion of the outward facing surface is covered in VELCRO loop covering.

Window 32 is an opening lying in the middle of the belt. Attachment features are provided around this opening in order to attach the cover panel to the belt. In the embodiment shown, tension strap 24 and tension strap 26 lie on the right side of window 32. Tension straps 28 and 30 lie on the left side of the window.

Pockets are preferably provided adjacent to the right and left edges of the belt. These assist the user in applying the belt—as will be explained subsequently. It is preferable to divide each pocket into a finger pocket and a thumb pocket. Thus, the pocket proximate right edge 80 is divided into right thumb pocket 16 and right finger pocket 18. Similarly, the pocket adjacent to left edge 78 is divided into left thumb pocket 20 and left finger pocket 22.

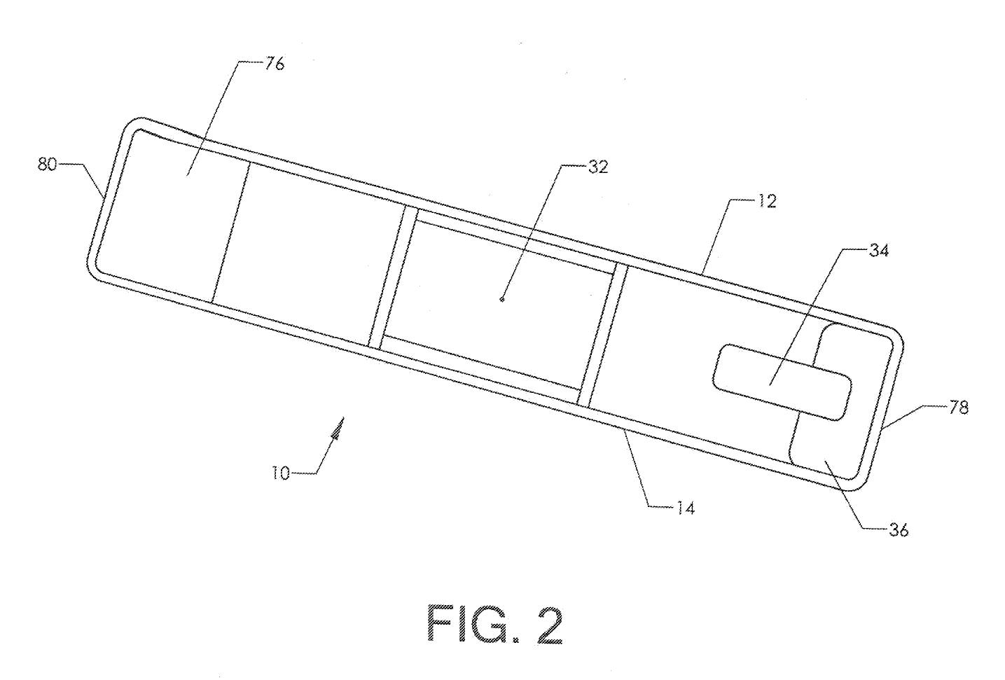

FIG. 2 shows the inward facing side of belt 10 (the side that would normally be placed against the patient). The inward facing side includes VELCRO hook panel 76 adjacent to right edge 80. An elongated loop panel 34 is provided proximate left edge 78. A 4-way stretch panel 36 is also provided adjacent to the left edge.

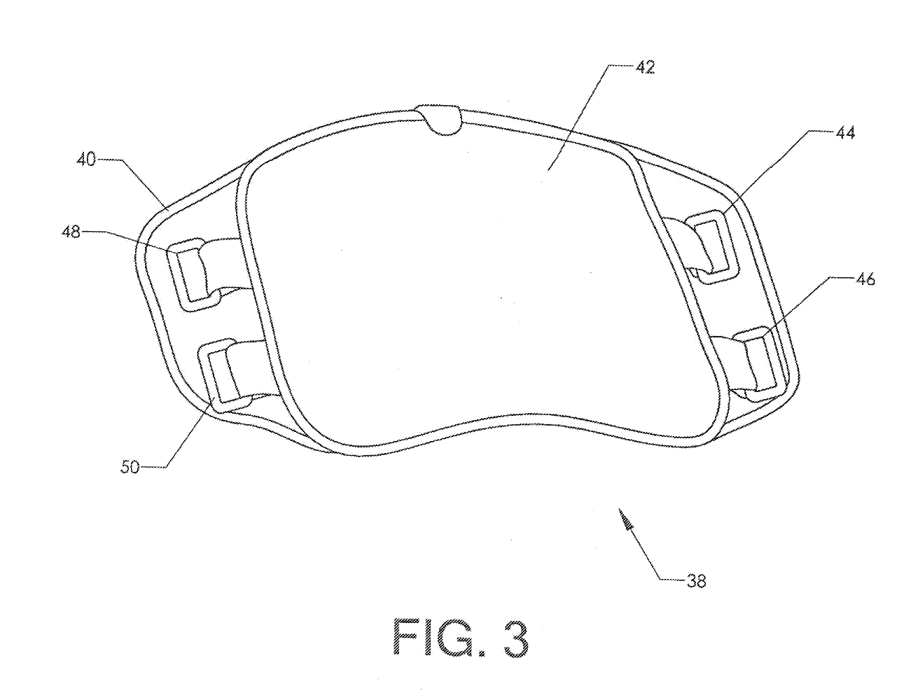

The second major component—cover panel 38—is shown in FIG. 3. The cover panel includes main body 40 and stay pocket cover 42. A pocket is formed between main body 40 and stay pocket cover 42.

Attachment features are provided to facilitate the attachment of the cover panel to the belt. In the embodiment shown in FIG. 3, four strap loops are attached to the cover panel (strap loops 44, 46, 48, and 50). These cooperate with the tensions straps 24, 26, 28, and 30 found on the belt.

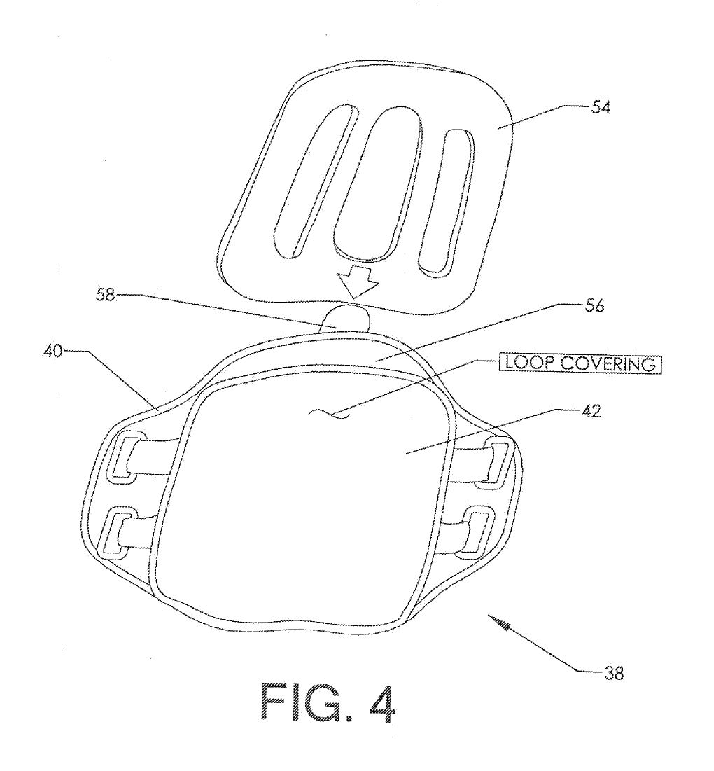

FIG. 4 is an exploded view. The pocket between main body 40 and stay pocket cover 42 may optionally be used to house him bar stay 54. Once the lumbar stay is inserted into the pocket through the top opening, hook tab 58 may be curled over the top and secured to the VELCRO loop covering on the exterior of stay pocket cover 42.

The lumbar stay is preferably a semi-rigid material such as a sheet of flexible plastic. It is preferably contoured to match the shape of a patient's lumbar area. The use of the stay helps conform cover panel 38 to the patient's anatomy. It is possible to provide a variety of different shapes for lumbar stay 54. It is also possible to provide an embodiment in which the lumbar stay may be selectively deformed by the clinician.

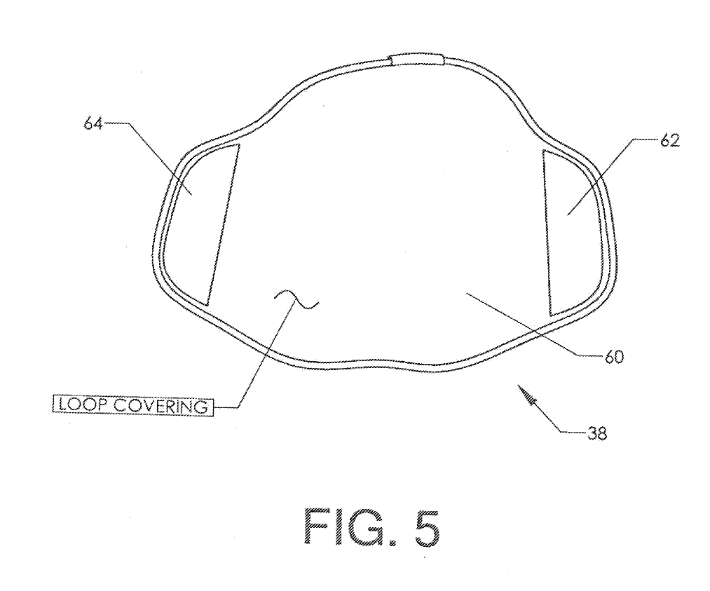

FIG. 5 shows the opposite side of cover panel 38. Inward facing surface 60 is the surface which will contact the patient. Although the tensions traps and strap loops are provided to secure the cover panel in position, it is preferable to provide another more temporary securing means. In the embodiment of FIG. 5, left hook panel 62 and right hook panel 64 are provided. These can be pressed against the loop covering on the outward facing side of the belt in order to hold the cover panel in position before the tension straps are suitably tightened.

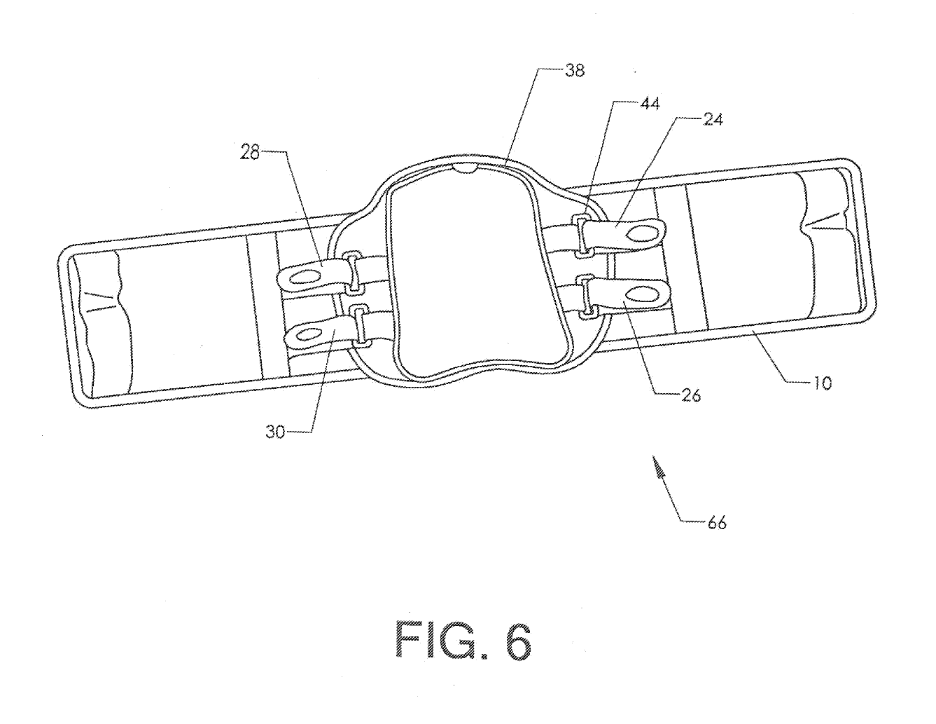

FIG. 6 shows cover panel 38 in place on belt 10 to form assembly 66. The cover panel is held in position using the VELCRO features described previously. The four straps attached to the belt are threaded through the four loops on the cover panel. As an example, Tension strap 24 is threaded through strap loop 44 and pressed back on itself. The free end of the tension strap preferably includes a VELCRO hook panel (facing away from the viewer in FIG. 6). This hook panel may be pressed back against the anchored portion of the strap (which includes a loop covering) or any other portion of the outward facing surface of the belt (which also includes a loop covering).

The assembly is thus united so that it may be handled as one unit and placed on the patient as one unit. However, the inclusion of the tension strap assemblies allows the assembly to be progressively tightened after it is in place (explained in detail subsequently).

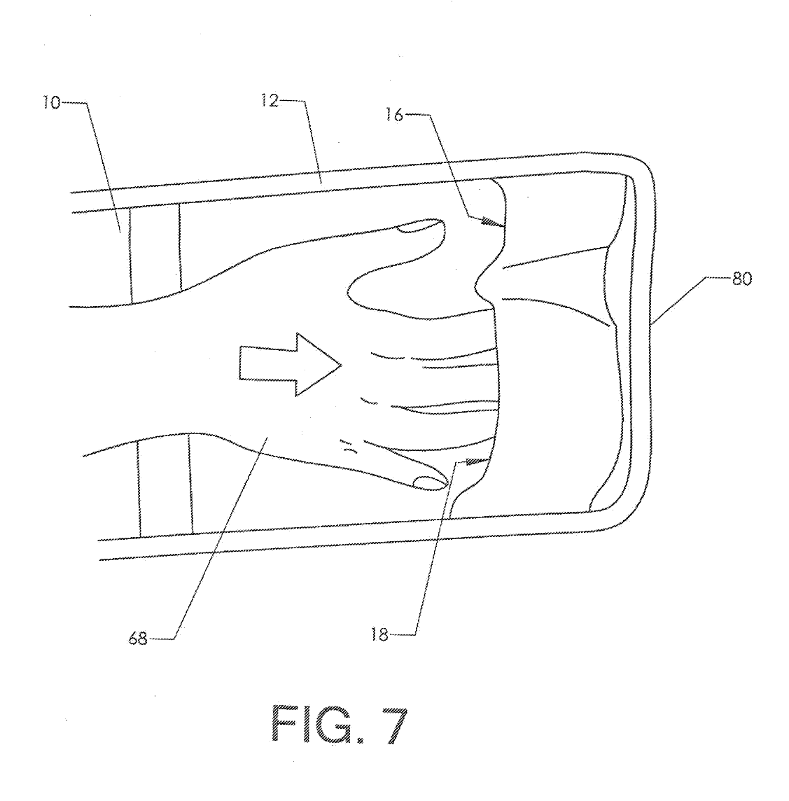

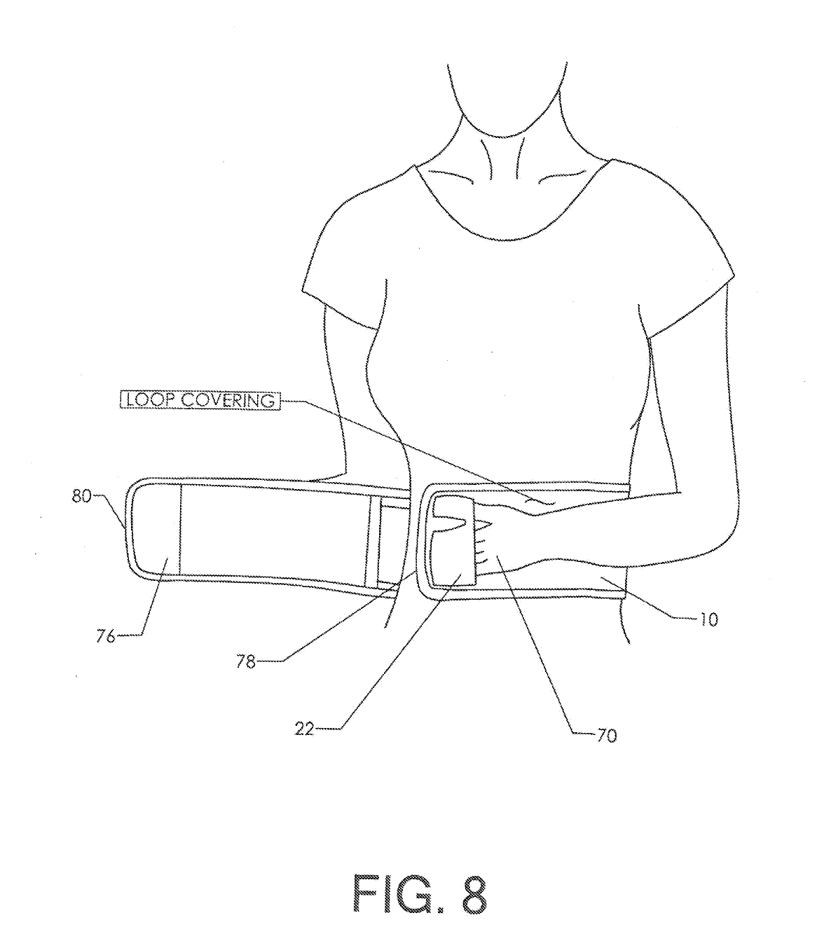

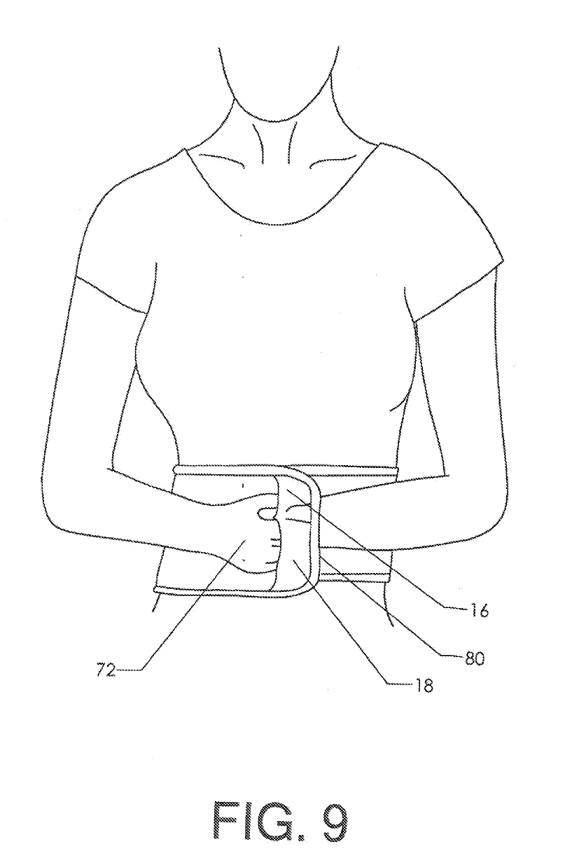

The reader will recall from the prior description that each side of the belt preferably includes a pocket to facilitate installation. FIG. 7 shows the right pocket in detail. In the preferred embodiments, the pocket is split into two portions—right thumb pocket 16 and right finger pocket 18. Hand 68 is inserted into the two pockets as shown. This feature allows the user to easily place the belt in the proper position—as is shown in FIGS. 8 and 9.

In FIG. 8, left hand 70 has been inserted into the pocket(s) proximate left edge 78. The right hand has been placed in the corresponding pockets proximate the right edge. The user places her left hand over her abdomen—holding the left portion of the belt as shown. The right hand is extended away from the body to hold right edge 80 clear. The reader will recall that the outward facing side of the belt is covered in VELCRO loop covering as shown. The outward facing side of the finger and thumb pockets is preferably also covered in loop material. Hook panel 76—on the right side of the belt—is facing inward.

If the user then brings her right hand in over her left, hook panel 76 will be pressed against the loop covering on the outward facing surface of the belt. (Including the outward facing surfaces of left thumb pocket 20 and left finger pocket 22). FIG. 9 shows this configuration. The user then withdraws both her hands outward from the pockets. The belt will remain in position, because of the hook and loop interlock.

The reader will therefore perceive that the belt is a band with a gap or break in its anterior portion which allows it to be opened and installed. A VELCRO link is preferably used to close the break. As shown in FIG. 9, the VELCRO link will usually attach the belt with the two edges overlapping for a considerable distance. The overlap may be varied in order to adjust the circumference of the belt to suit the needs of a particular user.

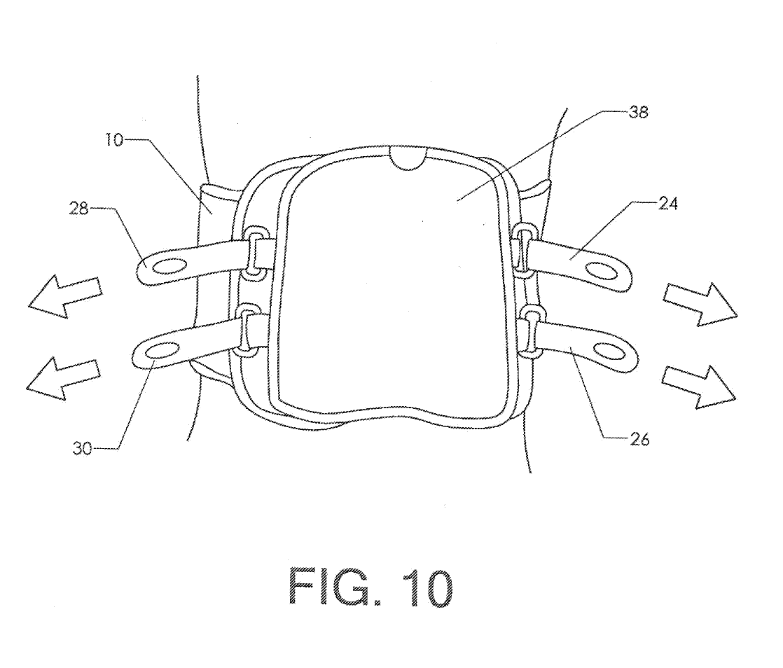

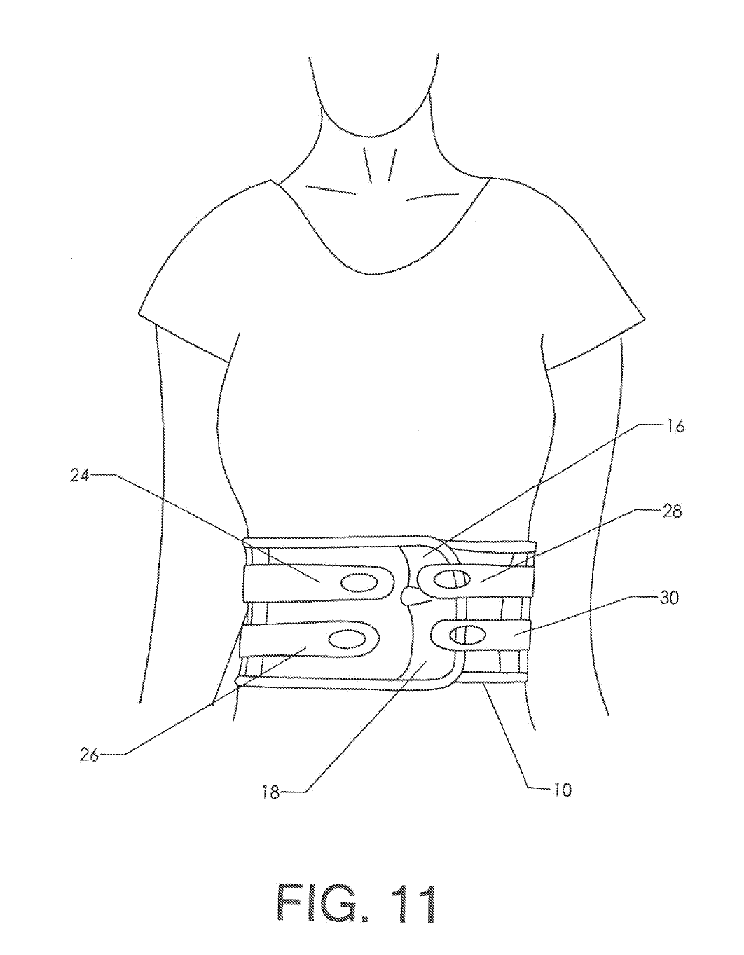

The cover panel will often be attached to the belt before the belt is placed on the user (though this need not always be the case). Once the belt is fastened as shown in FIGS. 8 and 9, the user will typically tighten the device to create the desired fit. FIGS. 10 and 11 show this portion of the installation process.

FIG. 10 shows the tightening of the tension straps. With the belt in place, each of the tension straps may be grasped and progressively tightened. The tension straps preferably include an elastic portion—or possibly the entire length of the strap is elastic—so that they may elongate as they are tightened. A gripping feature—such as an oval bole through the strap—is preferably provided on the end of each tension strap so that the user may more easily grasp the strap and pull.

FIG. 10 shows the patient from a vantage point behind the patient. The straps may be tightened by a clinician or by the patient (since the end of the straps lie adjacent to the patient's sides and may be easily reached by the patient).

FIG. 11 shows patient from the front, after the tightening process is completed. The tension straps preferably include a VELCRO hook portion on the side facing toward the belt. Thus, each strap may be fixed in a desired position by pressing the strap in against the belt (engaging the loop covering on the outward facing surface of the belt). It will then remain secure until the patient peels it loose. As shown in FIG. 11, the free end of each of the tension straps 24, 26, 28, and 30 has been secured to belt 10.

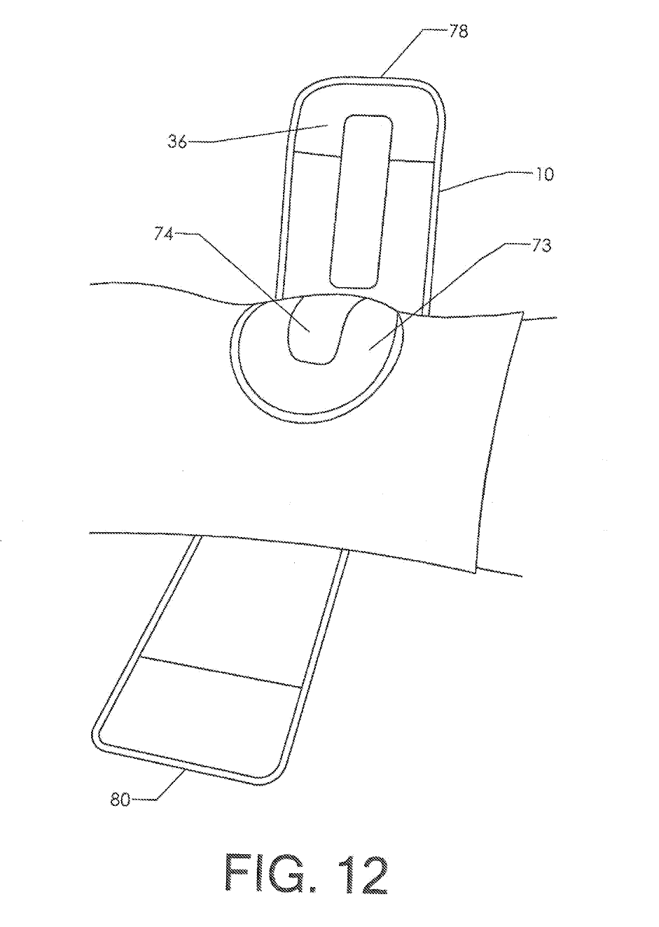

Other components may be selectively added to the belt assembly. FIG. 12 shows the use of an abdominal pad to spread the force of the belt across the patient's abdomen. Abdominal pad 73 is preferably provided in a variety of sizes and possibly shapes. A suitable version is placed on the patient's abdomen as shown. Abdominal pad 73 is provided with hook panel 74. Loop panel 34 is provided on belt 10 proximate left edge 78. Loop panel 34 is positioned to engage hook panel 74 when the belt is installed in the previously-described way (overlapping right edge 80 over left edge 78).

Abdominal pad 73 is thereby properly positioned with respect to the belt. Even if the belt is removed and reinstalled hook panel 74 will remain engaged to loop panel 34 and retain the proper position.

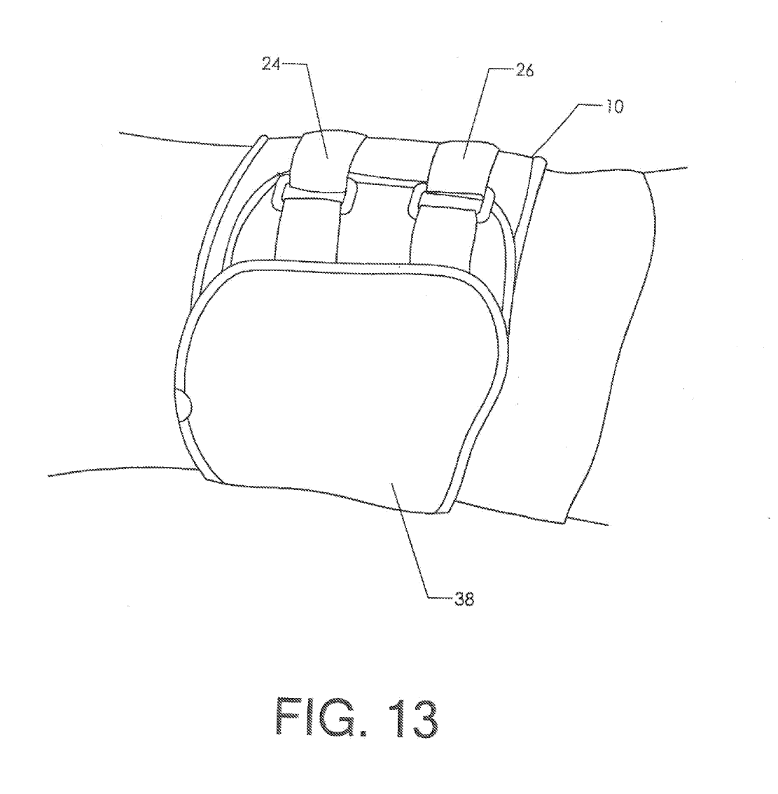

One of the advantages of the present invention is the fact that it allows cryo therapy cold packs to he added and/or exchanged without requiring the removal of the entire belt. FIGS. 13-16 illustrate this process. In FIG. 13 the belt and cover panel have already been applied to the patient. This configuration represents a circumstance where the practitioner wishes to add or exchange a cold pack. The patient has been rolled onto her left side to access cover panel 38.

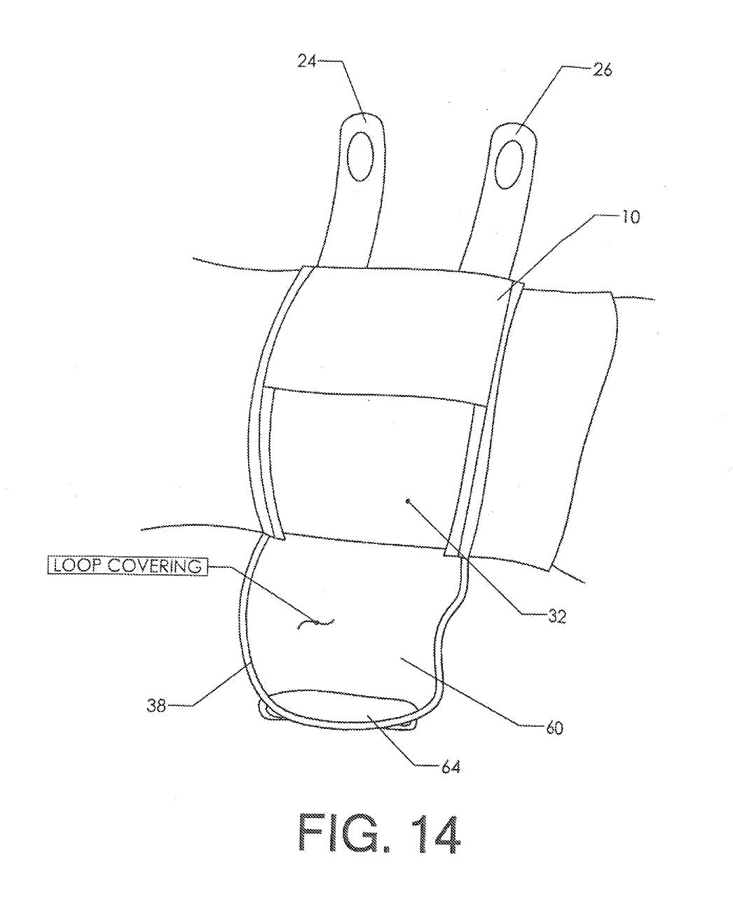

Tension straps 24 and 26 are disconnected from belt 10 so that the right edge of cover panel 38 can swing open. FIG. 14 shows tension straps 24 and 26 after they have been disconnected from cover panel 38. Right hook panel 64 has been pulled free of the loop material on the belt so that the cover panel can fall open as shown—revealing inward facing surface 60. As described previously, at least a portion of inward facing surface 60 is covered with VELCRO loop covering.

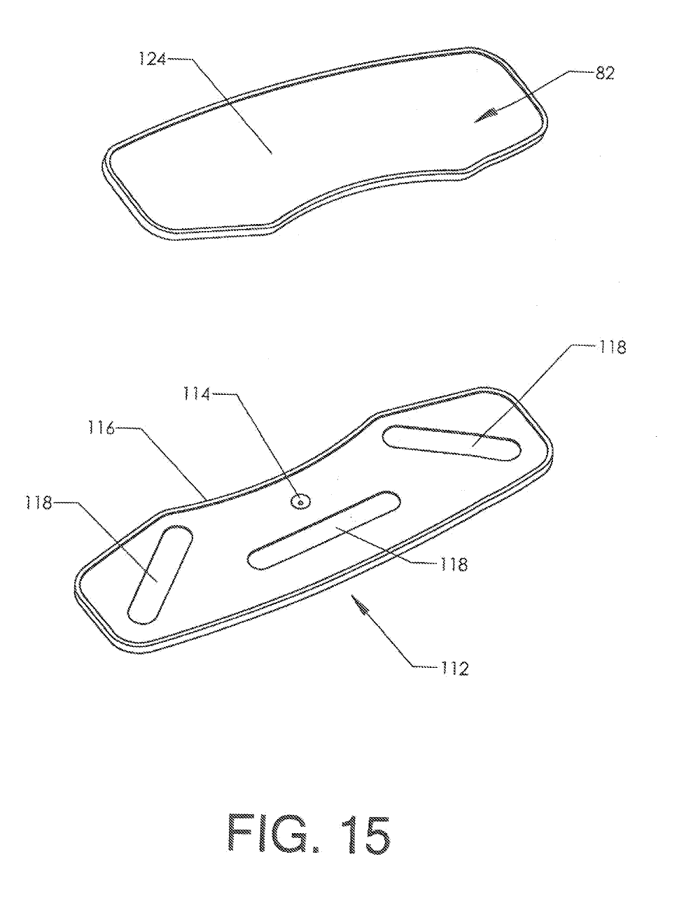

FIG. 15 shows two cold packs 112 which are suitable for application to the lumbar area. The two cold packs are identical. Each cold pack has two sides. Soft surface 82 is a smooth surface which may be placed directly against the patient's skin. This surface is facing upward for the cold pack illustrated near the top of FIG. 15.

The opposite surface is intended to face away from the patient. It includes one or more hook panels 118. This surface is facing upward for the cold pack illustrated near the bottom of FIG. 15. The cold packs may include other features—such as relief 116 which allows it to better conform to portions of a patient's anatomy.

In the preferred embodiment, each cold pack includes vent 114. The cooling medium is preferably one which forms a highly ordered crystalline structure upon freezing—much like snow. The freezing process therefore requires the volume within the cold pack to expand. Vent 114 admits air during the freezing process so that the volume inside the cold pack can freely expand. Vent 114 allows the escape of air as the cooling medium returns to a liquid.

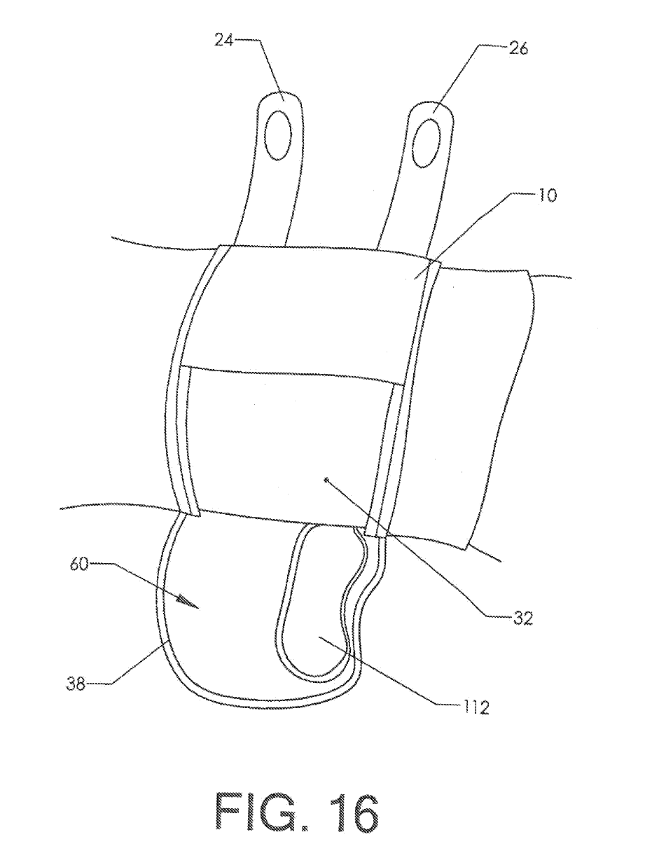

Turning to FIG. 16, the application of a cold pack to the belt assembly will be explained. The user places a cold pack 112 on inward facing surface 60 of cover panel 38 (with the surface including hook panels 118 pressing against inward facing surface 60). The cold pack becomes attached to the cover panel by the hook panels 118 on the cold pack locking to the VELCRO loop covering on inward facing surface 60 of cover panel 38. The use of the VELCRO attachment allows the cold pack to be placed in many different positions. The reader will also observe that sufficient space is provided for two or more cold packs.

Once the cold pack is in the desired position, the user closes window 32 by lifting cover panel 38 back into position. Right hook panel 64 will engage the loop covering on the belt and hold the panel in place. The user may then reattach and tighten tension straps 24 and 26 to complete the process.

Those skilled in the art will realize that the steps thus described allow the user to quickly add or swap cold packs. During cryo therapy to the lumbar region, it will be necessary to swap cold packs approximately every four hours. Using the present invention, the swap may be made without removing the belt. The user simply opens the window, peels away the existing cold packs, places new cold packs, and closes the window.

Those skilled in the art will also realize that the convenient access to the lumbar region allows a wound dressing to be more rapidly inspected and changed. It also facilitates the addition of other treatment devices, such as a TENS unit.

The belt remains around the patient's abdomen throughout this process. The belt's presence provides structural reinforcement, which is particular important in cases where the lumbar fascia has been incised. It is important to limit motion in such cases. The cover panel not only retains the cold packs, it also provides a significant retention force across the patient's lower back.

Although the preceding descriptions present considerable detail they should be properly viewed as illustrating preferred embodiments of the present invention rather than limiting the scope of the invention. Many more embodiments following the same principles will occur to those skilled in the art. As a first example, the order of the steps presented in the descriptions of how the belt is applied and removed are generally unimportant. One may choose to attach the cover panel prior to applying the belt to the patient. One may also choose to attach the cover panel after the belt is in place. As a second example, snaps or buckles could be substituted for the hook-and-loop attachments described. As a third example, one side of the cover panel could be permanently attached to the belt, leaving the other side to swing open. Accordingly, the scope of the invention should be fixed by the following claims rather than by the examples given.

1. A method for applying cryo therapy to a patient's lumbar region, comprising:
a. providing a belt having a top edge, a bottom edge, a right edge, and a left edge;
b. said belt opening into a window between said right and left edges;
c. providing a VELCRO link for attaching a portion of said belt proximate said right edge to a portion of said belt proximate said left edge, with said right and left edges overlapping;
providing a cover panel, with said cover panel being sized to cover said window;
e. said cover panel including an inward facing surface, with said inward facing surface being covered in VELCRO loop material;
f. providing a cold pack containing a cooling medium;
g. said cold pack including a hook panel;
h. applying said belt to said patient with said window being positioned over said patient's lumbar region;
i. attaching said cover panel to said belt so that said cover panel lies over said window;
j. detaching at least a portion of said cover panel from said belt in order to pull said at least a portion of said cover panel away from said belt, thereby revealing said inward facing surface of said cover panel;
k. attaching said cold pack to said inward facing surface of said cover panel by pressing said hook panel of said cold pack against said VELCRO loop material on said inward facing surface; and
reattaching said at least a portion of said cover panel to said belt, thereby placing said cold pack in contact with said patient's lumbar region.

2. A method for applying cryotherapy as recited in claim 1, wherein:
a. said belt includes an outward facing surface covered in VELCRO loop material;
b. said cover panel includes a right hook panel and a left hook panel; and
c. said cover panel is attached to said belt by pressing said right and left hook panels on said cover panel against said VELCRO loop panel on said belt.

3. A method for applying cryotherapy as recited in claim 2, further comprising:
a. wherein said cover panel includes a right side and a left side;
b. a first tension strap adjustably linking said right said of said cover panel to said belt; and
c. a second tension strap adjustably linking said left side of said cover panel to said belt.

4. A method for applying cryotherapy as recited in claim 3, wherein:
a. a first end of said first tension strap is attached to said belt, said first tension strap loops through a strap loop on said right side of said cover panel, and said second end of said first tension strap attaches to said belt; and
b. a first end of said second tension strap is attached to said belt, said second tension strap loops through a strap loop on said left side of said cover panel, and said second end of said second tension strap attaches to said belt.

5. A method for applying cryotherapy as recited in claim 3, wherein said first and second tension straps are elastic.

6. A method for applying cryotherapy as recited in claim 1, wherein:
a. said belt includes a right pocket proximate said right edge; and
b. said belt includes a left pocket proximate said left edge.

7. A method for applying cryotherapy as recited in claim 1, wherein said cover panel includes a lumbar stay.

8. A method for applying cryo therapy to a patient's lumbar region, comprising:
a. providing a belt having a gap and a window opposite said gap;
b. providing a VELCRO link for closing said gap and linking said belt into a continuous circle;
c. providing a cover panel, with said cover panel being sized to cover said window;
d. said cover panel including an inward facing surface, with said inward facing surface being covered in VELCRO loop material;
e. providing a cold pack containing a cooling medium;
f. said cold pack including a hook panel;
g. applying said belt to said patient with said window being positioned over said patient's lumbar region;
h. attaching said cover panel to said belt so that said cover panel lies over said window;
i. uncovering said window by detaching at least a portion of said cover panel from said belt, thereby revealing said inward facing surface of said cover panel;
j. attaching said cold pack to said inward facing surface of said cover panel by pressing said hook panel of said cold pack against said VELCRO loop material on said inward facing surface; and
k. recovering said window by reattaching said at least a portion of said cover panel to said belt, thereby placing said cold pack in contact with said patient's lumbar region.

9. A method for applying cryotherapy as recited in claim 8, wherein:
a. said belt includes an outward facing surface covered in VELCRO loop material;
b. said cover panel includes a right hook panel and a left hook panel; and
c. said cover panel is attached to said belt by pressing said right and left hook panels on said cover panel against said VELCRO loop panel on said belt.

10. A method for applying cryotherapy as recited in claim 9, further comprising:
a. wherein said cover panel includes a right side and a left side;
b. a first tension strap adjustably linking said right said of said cover panel to said belt; and
c. a second tension strap adjustably linking said left side of said cover panel to said belt.

11. A method for applying cryotherapy as recited in claim 10, wherein:
a. a first end of said first tension strap is attached to said belt, said first tension strap loops through a strap loop on said right side of said cover panel, and said second end of said first tension strap attaches to said belt; and
b. a first end of said second tension strap is attached to said belt, said second tension strap loops through a strap loop on said left side of said cover panel, and said second end of said second tension strap attaches to said belt.

12. A method for applying cryotherapy as recited in claim 10, wherein said first and second tension straps are elastic.

13. A method for applying cryotherapy as recited in claim 8, wherein:
a. said belt includes a right pocket proximate said right edge; and
b. said belt includes a left pocket proximate said left edge.

14. A method for applying cryotherapy as recited in claim 8, wherein said cover panel includes a lumbar stay.

15. A method for applying cryo therapy to a patient's lumbar region, comprising:
a. providing a circular belt with an adjustable opening and a window opposite said adjustable opening;
b. providing a VELCRO link for closing said adjustable opening;
c. providing a cover panel, with said cover panel being sized to cover said window;
d. said cover panel including an inward facing surface, with said inward facing surface being covered in VELCRO loop material;
e. providing a cold pack containing a cooling medium;
f. said cold pack including a hook panel;
g. applying said belt to said patient by looping said belt around said patient and closing said adjustable opening, with said window being positioned over said patient's lumbar region;
h. attaching said cover panel to said belt so that said cover panel lies over said window;
i. detaching at least a portion of said cover panel from said belt in order to pull said at least a portion of said cover panel away from said belt, thereby revealing said inward facing surface of said cover panel;
j. attaching said cold pack to said inward facing surface of said cover panel by pressing said hook panel of said cold pack against said VELCRO loop material said inward facing surface; and
k. reattaching said at least a portion of said cover panel to said belt, thereby placing said cold pack in contact with said patient's lumbar region.

16. A method for applying cryotherapy as recited in claim 15, wherein:
a. said belt includes an outward facing surface covered in VELCRO loop material;
b. said cover panel includes a right hook panel and a left hook panel; and
c. said cover panel is attached to said belt by pressing said right and left hook panels on said cover panel against said VELCRO loop panel on said belt.

17. A method for applying cryotherapy as recited in claim 16, further comprising;
a. wherein said cover panel includes a right side and a left side;
b. a first tension strap adjustably linking said right said of said cover panel to said belt; and
c. a second tension strap adjustably linking said left side of said cover panel to said belt.

18. A method for applying cryotherapy as recited in claim 17, wherein:
a. a first end of said first tension strap is attached to said belt, said first tension strap loops through a strap loop on said right side of said cover panel, and said second end of said first tension strap attaches to said belt; and
b. a first end of said second tension strap is attached to said belt, said second tension strap loops through a strap loop on said left side of said cover panel, and said second end of said second tension strap attaches to said belt.

19. A method for applying cryotherapy as recited in claim 17, wherein said first and second tension straps are elastic.

20. A method for applying cryotherapy as recited in claim 15, wherein:
a. said belt includes a right pocket proximate said right edge; and
b. said belt includes a left pocket proximate said left edge.
